(19) United States
(12) Patent Application Publication
(10) Pub. No.: US 2008/0220088 A1
(43) Pub. Date: Sep. 11, 2008

(54) Composition for Treating Wounds and Burns

(75) Inventors: Changoer Lekhram, Ijsselstein (NL); Willem Frederik Van Den Bosch, Oegstgeest (NL); Hendrik Jan Cornelis Meijerink, Brussel (BE)

Correspondence Address: FOLEY AND LARDNER LLP;SUITE 500, 3000 K STREET NW, WASHINGTON, DC, 20007, US

(73) Assignee: NGEN PHARMACEUTICALS N.V., Kaya Richard Ji Beaujon z/n, Curacao (AN)

(21) Appl. No.: 11/576,878

(22) PCT Filed: Oct. 7, 2005
(86) PCT No.: PCT/NL05/00729

§ 371 Date: Aug. 28, 2007

Related U.S. Application Data
(60) Provisional application No. 60/616,231, filed on Oct. 7, 2004.

(51) Int. Cl. A61K 33/40; A61K 33/00; A61K 33/22

(52) U.S. Cl. 424/613; 424/600; 424/660

(57) ABSTRACT

The invention discloses the use of a liquid composition, capable of promoting the growth of tissue cells at the site of a wound, a process that promotes regeneration of tissue and wound healing. More particularly, the present invention relates to the use of a liquid composition comprising a component (a) selected from the salts consisting of cations Aₙ⁺ and anions derived from halogen oxides according to the general formula [OₘX]⁻, a component (b) selected from the group of oxygen donors, and a component (d) selected from the group of liquid binders for the treatment of open wounds and burns.

### FIELD OF THE INVENTION

This invention concerns the use of a liquid composition, capable of promoting the growth of tissue cells at the site of a wound, a process that promotes regeneration of tissue and wound healing. More particularly, the present invention relates to the use of a liquid composition comprising a component (a) selected from the salts consisting of cations Aₙ⁺ and anions derived from halogen oxides according to the general formula [OₘX]⁻, a component (b) selected from the group of oxygen donors, and a component (d) selected from the group of liquid binders for the treatment of open wounds and burns.

### BACKGROUND OF THE INVENTION

The process of healing of open wounds and burns is a very complex, not fully understood process. Nowadays the most common means of treating open wounds or burns mainly comprise temporary wound closure/covering for protection against e.g. moisture loss and infection using autografts, dermal replacement products or hydrogels such as alginates, administration of substances for cooling and pain relief, and promoting wound healing by administering drugs which are usually hemostatic for suppressing bleeding in the wound region, anti-inflammatory for suppressing inflammation, sterilizers for sterilisation so as to prevent miscellaneous bacteria from invading the wound, or drugs with a combination of the above pharmaceutical effects. However, at present “reconstruction of tissue” as the final stage of wound healing has to rely on the auto-therapy inherent in living bodies.

Open wounds or burns that have impaired blood flow are often characterised by a delay or complete failure of the healing process. This is ascribed to the fact that in hypoxic tissue resistance to infection is lower and the fact that the process of auto-therapy is compromised. Wound oxygenation therefore has long been known to be beneficial in the healing of (chronic) wounds and burns. The healing of wounds and the effect of oxygen thereon has been intensively studied, a useful summary is presented in J. D. Whitney, “Physiological Effects of Tissue Oxygenation on Wound Healing”, HEART & LUNG, September 1989, Vol. 18 No. 5, pp. 466-474.

Supplying an environment, rich in molecular oxygen, to the open wounds or burns, is believed to be beneficial in wound healing by activating the cells present at the periphery of wound sites by promoting their metabolism and by stimulating phagocytosis and killing of bacteria by neutrophils or polymorphonuclear cells (PMNs), which involves the production of oxygen radicals and superoxides and is directly influenced by the oxygen concentration in the tissue.

Active oxygen is quite different in view of chemical species and biochemical activities from ordinary molecular oxygen. While ordinary oxygen contributes only to metabolism but does not function as bio-signals for the growth of the cells, the active oxygen does not contribute to the metabolism but function as bio-signals for the growth of the cells.

In auto-therapy, the reconstruction of the tissues at the wound site is conducted through the following processes: (1) macrophages gathering at the wound site yield growth factors and enzymes, (2) the growth factors and the enzymes are activated, (3) the activated growth factors and enzymes stimulate increase or movement of fibroblasts to grow the cells, and (4) the tissue is reconstructed (regenerated) by the grown new cells.

In the case of auto-therapy, macrophages gathering at the wound sites produce active oxygen and the active oxygen functions as bio-signals to activate the growth factors and the enzymes yielded also from the macrophage. That is, it is known that active oxygen contributes to activation of growth factors and enzymes in the process of auto-therapy. Active oxygen supplied from the outside activates the growth factors and the enzymes together with active oxygen produced spontaneously from macrophage and promotes the growth of cells by a process similar to that of auto-therapy. As a result, growth of the cells is further promoted and the tissues at the wound site are reconstructed (regenerated) in a shorter period of time.

The importance of both ordinary molecular oxygen and of activated oxygen in these processes has once more been demonstrated by Cho et al. (Am. J. Physiol. Heart Circ. Physiol., 280; 2001), particularly in the process of angiogenesis. Their findings suggest that activated oxygen stimulates macrophages to release higher levels of VEGF (vascular endothelial growth factor) and can thereby drive angiogenesis in wounds. They found that neutrophils release higher amounts of activated oxygen when exposed to hyperoxia in vitro, and that VEGF itself is stimulated by hyperoxia in wound cylinders.

A plurality of methods for supplying either normal molecular oxygen or active oxygen to open wounds or burns is generally known in the art.

U.S. Pat. No. 5,865,722 discloses a “topical” hyperbaric oxygen chamber which is a special type of the long known hyperbaric oxygen chamber. Hyperbaric oxygen chambers are for the purpose of introducing of pressurized molecular oxygen into an encapsulated environment with this oxygen to promote the healing of various types of wounds. Specifically it was mentioned that the treatment of open wounds within a hyperbaric oxygen chamber promotes healing and suppresses bacterial infection. “Topical” hyperbaric oxygen chambers have been proposed due to the expenses of large hyperbaric oxygen chambers and due to the risks of systemic oxygen toxicity thereof. These topical hyperbaric oxygen chambers, however, have the disadvantage of difficulties in air-tight sealing to the body without interfering with blood supply, resulting in operation under modestly elevated pressure, while consuming large amounts of oxygen, because of leakage.

U.S. Pat. No. 5,792,090 discloses a more convenient means of increasing the wound oxygen tension, through the application of an oxygen generating wound dressing which renewably and non-sustainingly chemically generates oxygen. The wound dressing contains an aqueous liquid, containing hydrogen peroxide, in an oxygen supply solution reservoir, which also contains a solid hydrogen peroxide decomposition catalyst, such as manganese dioxide, the system thus being capable of supplying molecular oxygen through chemical reaction. U.S. Pat. No. 6,139,876 discloses a gel such as a gelatine that can be saturated with oxygen, basically by heating it until the gelatine is a liquid, and introducing, in a closed vessel, oxygen gas into it so that the pressure is at least about 0.15 Mpa and mixing the liquid with oxygen; cooling it to enable a gelatine to form having excess oxygen therein in the form of microscopic bubbles. This oxygen containing gelatine can be applied to open wounds and burns for supplying molecular oxygen to said wound or burn. The major disadvantage of this method is that the gelatine has to be stored in a special container, wherein the pressure is maintained above atmospheric pressure during storage.

According to US 2002/0160053 A1 application of a solution containing activated oxygen can promote growth of new tissue cells at wound sites, by enhancing certain biochemical reactions in and around the region of the wound, by activation of the growth factors and enzymes, similar to the process of auto-therapy.

The invention, as disclosed in US 2002/0160053 A1, allows the aforementioned object to be achieved by producing an aqueous solution, which comprises water containing active oxygen and halogen ions. The active oxygen can include singlet oxygen (¹O₂) formed by excitation of triplet oxygen, superoxide (O₂⁻) formed by reduction of oxygen by a single electron, and hydroxy radical (HO.), as well as hypochlorous ions (ClO⁻) and peroxy radicals (ROO.), alkoxy radicals (RO.), and hydroperoxides (ROOH).

A major drawback of the invention, according to US 2002/0160053 A1, is the fact that the solution, containing activated oxygen, has to be obtained by electrolysis, which is to be performed, according to the inventors, by the hospitals themselves, which is inconvenient and moreover suggests that these solutions are not very stable. Also, trained personnel is required to operate the electrolysis apparatus.

U.S. Pat. No. 4,507,285 discloses aqueous compositions containing stabilised activated oxygen in a matrix of chlorite ions and pharmaceutical compositions, which may be applied for the treatment of skin diseases or for healing wounds and burns. The stabilised activated oxygen is present in the form of an aqueous solution. The production of the composition according to this invention involves many long (4 weeks!) and laborious reaction steps, which is clearly an inconvenient and costly process.

U.S. Pat. No. 6,488,965 discloses a liquid preparation that is said to be suitable for the treatment of wounds and burns, said preparation comprising a metal chlorite and a peroxide compound, preferably hydrogen peroxide. The preparation may be in the form of a gel, cream or ointment. Successful treatments of wounds and burns are, however, not disclosed.

There is thus still a need for a medicament for treatment of open wounds and burns, capable of promoting wound healing through the mechanism of auto-therapy, most preferably by supplying oxygen to the wound, either ordinary molecular oxygen or activated oxygen, which is convenient to use and easily and cost-effectively stored and produced.

U.S. Pat. No. 6,017,515 discloses a combination preparation for bleaching teeth or treating skin complaints and mucous membrane disorders, especially lesions, said combination preparation being able, when mixed and applied topically, to deliver chemically generated oxygen. The composition according to U.S. Pat. No. 6,017,515 comprises a component (a) selected from the salts consisting of cations Aₙ⁺ and anions derived from halogen oxides according to the general formula [OₘX]⁻, and a component (b) selected from the salts consisting of cations Aₙ⁺ and anions derived from borates according to the general formula [BₚO_{q}]^{r-}, wherein A is a metal selected from Groups 1 or 2 of the Periodic System of the Elements, X is a halogen atom, m=1-4, n=1 or 2, p=1-4, q=1-8, and r=1-3.

### SUMMARY OF THE INVENTION

We have surprisingly found that the active components from the invention disclosed in U.S. Pat. No. 6,017,515 can be used successfully for promoting the healing of open wounds and burns by, without being bound to theory, chemically releasing oxygen. A composition containing these components can be used for the manufacture of a medicament that, when applied topically, promotes the healing of open wounds and burns. In a preferred embodiment of the present invention this composition can be provided with an oxygen donor stabilising agent.

### DETAILED DESCRIPTION OF THE INVENTION

One aspect of the present invention relates to the use of a liquid composition for the treatment of open wounds and burns, wherein the composition comprises a component (a) selected from the salts consisting of cations Aⁿ⁺ and anions derived from the halogen oxides according to the general formula [OₘX]⁻, wherein A is a metal selected from Groups 1 and 2 of the Periodic System of the Elements, X is a halogen atom, m=1-4, n is 1 or 2, and a component (b) selected from the group of oxygen donors, and a component (d) selected from the group of liquid binders. According to the present invention, the liquid composition is obtainable by combining at least components (a), (b) and (d).

In the formula [OₘX]⁻ X can be fluorine, chlorine, bromine or iodine, preferably chlorine. Examples of the anion [OₘX]⁻ are the hypochlorite, hypoiodite, chlorite, iodite, chlorate, bromate, iodate, perchlorate and periodate anions. According to the present invention it is preferred that m=1, so that the anion [OₘX]⁻ is most preferably the hypochlorite anion.

Component (b) is preferably selected from oxygen donors selected from the group consisting of metal borate compounds, metal peroxide compounds, metal percarbonate compounds (also known as carbonate peroxyhydrate compounds), metal persulfate compounds (also known as peroxosulfur compounds), metal perphosphate compounds, wherein the metal is an alkaline or alkaline-earth metal, halogen oxide compounds, hydrogen peroxide, and organic peroxides. More preferably, component (b) is selected from the group consisting of metal perborate compounds (also known as peroxoborate compounds), metal percarbonate compounds, metal peroxide compounds, hydrogen peroxide, halogen oxide compounds and organic peroxides.

According to the invention, the halogen oxide compounds are preferably formed in situ from halogen oxide compound precursors. Suitable halogen oxide compound precursors are for example metal halite such as sodium chlorite and metal hypohalite such as sodium hypochlorite compounds as is well known in the art.

According to the invention, the perborate compounds are preferably selected from the salts consisting of cations Aⁿ⁺ and anions derived from borates according to the general formula [BₚO_{q}]^{r-}, wherein A is a metal selected from Groups 1 or 2 of the Periodic System of the Elements, and p=1-4, q=1-8, and r=1-3. Examples of the anion [BₚO_{q}]^{r-} are perborate (BO₃)⁻, metaborate (BO₂)⁻, orthoborate (BO₃)³⁻, hypoborate (B₂O₄)²⁻ and pyroborate or tetraborate anions (B₄O₇)²⁻. Preferably, p=1, q=2 or 3 and r=1. Most preferably, p=1, q=3 and r=1 which implies that the most preferred anion is perborate. According to the invention, the percarbonate compounds are preferably selected from the salts consisting of cations Aⁿ⁺ and anions CO₃²⁻. This implies that the percarbonates do not contain C—O—O-groups (the latter being known as peroxocarbonates). Additionally, the peroxosulfur compounds are preferable selected from peroxomonosulphates and peroxodisulphates. The metal peroxide compounds are preferably selected from alkaline-earth metal peroxide compounds, in particular calcium peroxide and magnesium peroxide. The organic peroxide is preferably carbamide peroxide. The halogen oxide is preferably chlorine dioxide ClO₂.

According to the invention, A is preferably selected from the group consisting of lithium, sodium, potassium and calcium and is most preferably sodium. Consequently, component (a) is most preferably sodium hypochlorite and component (b) is most preferably sodium perborate.

Components (a) and (b) can contain one or more molecules of water as water of crystallisation. However, for component (a) preferably an aqueous solution thereof is used. Component (b) can also contain one or more molecules of water as water of crystallisation, for example the monohydrates, dihydrates, trihydrates and tetrahydrates. According to the invention, all hydrates of both components (a) and component (b) can be used. If component (a) or component (b) occur in different polymorphs, all these polymorphs can be used in the present invention.

For the sake of clarity, if component (b) is for example sodium perborate tetrahydrate, the compound Na₂BO₃.4H₂0 is intended which at present is more often defined as Na₂[B₂(O₂)₂(OH)₄].6H₂O; the common name of the later compound is sodium peroxoborate hexahydrate. Likewise, if component (b) is for example sodium perborate monohydrate, the compound Na₂BO₃.H₂0 is intended which at present is more often defined as Na₂[B₂(O₂)₂(OH)₄]. Reference is made to Kirk-Othmer, Encyclopedia of Chemical Technology, 4^{th} Edition, Volume 18, pages 202-229 (1996) for the nomenclature of such compounds.

The present invention relates to the manufacture of a medicament, using a liquid composition comprising a component (a) and a component (b) as defined above. The term “liquid”, as used herein, refers to any non-solid pharmaceutical formulation known in the art, which is suitable for topical application. In particular the present invention relates to a gel, cream, paste or ointment or a more liquid material such as a suspension, dispersion or emulsion.

The composition is used for the manufacture of a medicament, which can be any type of medicament for topical application known in the art. Particularly preferred medicaments, according to the present invention, are a wound gel, a wound spray, or a wound dressing. The use of component (a) or component (b) in the manufacture of any medicament, which is intended to be used for treatment of open wounds or burns by combined application of component (a) and component (b) falls within the scope of the present invention.

The term “wound dressing”, as used herein, particularly refers to any material applied to a wound for protection, absorbance, drainage, etc. Numerous types of dressings are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer). According to one embodiment of the present invention, the medicament is such a wound dressing, which is impregnated or coated with the liquid, wound healing composition of the present invention.

In a particularly preferred embodiment a wound dressing is used that disintegrates and/or dissolves during contact with the wound or burn. In such an embodiment the dressing, which is impregnated or coated with the wound healing composition, is composed of a water-soluble polymer. Examples of such polymers that can be suitably used in the present invention are poly(lactic acid), poly(acrylic acid), and polyvinyl pyrrolidone.

The present invention relates to the manufacture of a medicament, using a composition, which comprises a component (a), a component (b) and a component (d), which medicament is able to supply an amount of chemically generated oxygen to the open wound or burn, which is effective in promoting wound healing. Preferably said medicament comprises from about 0.1% to about 30% by weight of the total composition of component (a), more preferably from about 0.5% to about 25%, most preferably from about 1.0% to about 20.0% by weight. The medicament of the present invention preferably comprises from about 0.1% to about 8.0% by weight of the total composition of component (b), more preferably from about 0.2% to about 6.0%, most preferably from about 0.3% to about 3.0% by weight, calculated on the total weight of the composition. The medicament further comprises from about 1.0% to about 80.0% by weight of the total composition of component (d), preferably from about 2.0% to about 50.0%, most preferably from about 3.0% to about 20.0% by weight.

The liquid binder according to the invention is used in particular for dispersing the components (a) and (b) and optionally (c), as explained below, and for enhancing the stability of the composition. Moreover, the liquid binder is used to adjust the concentrations of the active ingredients of the composition according to the invention. Obviously, the liquid binder has also additional properties, e.g. thickening properties, stabilising properties, water-binding promoting properties as is well known to the person skilled in the art. These liquid binders are preferably selected from the liquid polyols, polymeric binders, fumed silica and gums or a combination thereof. Examples of suitable liquid polyols include glycerol, propylene glycol, polyethylene glycol (PEG). Examples of suitable gums include natural gums and modified (semi-synthetic) gums, for example acacia gum, gum arabic, caraya gum, gum tragacanth, xanthan gum and cellulose gum. Examples of suitable polymeric binders are polyvinyl pyrrolidone, casein or salts thereof, wherein the salts comprise a metal of Group 1 or Group 2 of the Periodic System. According to the invention, it is preferred that the liquid binder is glycerol, glycol, propylene glycol, PEG, fumed silica, a gum, or a combination thereof. In a particularly preferred embodiment of the present invention the liquid binder is a combination of a liquid polyol and a fumed silica, most preferably PEG 1500 in combination with fumed silica, in total amounts of 0.005 to 4% and 1 to 20%, respectively, based on the total weight of the composition. Most preferred is an amount of PEG 1500 from about 0.01% to about 2% by weight of the total composition, and an amount of fumed silica from about 3% to about 10% by weight of the total composition. Moreover, component (a) is preferably employed as an aqueous solution comprising the binder, said aqueous solution comprising 25-75% by weight, preferably 35-65% by weight of the binder, calculated on the basis of the total weight of the aqueous solution.

Additionally, according to the invention the pH of the composition is essential for a controlled and long lasting release of the active component, i.e. oxygen. Tests have revealed that the pH is preferably in the range of 4-8, preferably 4.5-7.5 and most preferably 5.0-7.5.

The composition according to the present invention, which is used for the treatment of open wounds and burns, may further comprise a gelatinous thickener. Typically a cellulose material, such as cellulose, sodium carboxymethylcellulose, (hydroxy)propylcellulose, methylcellulose, or ethylcellulose, is used as a thickener. Preferably sodium carboxymethylcellulose is used in the present invention, in an amount of 0.2 to 4.0 percent by weight, preferably 0.5 to 2.5 percent by weight, calculated on the total weight of the composition.

The composition may further comprise an agent that counteracts loss of moisture, and that optionally also has an anti-microbial action. Preferably a carbohydrate, more preferably an alditol, such as, for example, erythritol, arabinitol, xylitol, galacitol, sorbitol, iditol, mannitol, hepitol, or octitol, is used as the agent that counteracts the loss of moisture. In the present invention the use of alditol is preferred, typically in an amount of 0.5 to 10.0 percent by weight preferably in an amount of 1.0 to 5.0 percent by weight, calculated on the total weight of the composition.

Preferably the compositions according to the present invention contain an anti-oxidant. Examples of suitable anti-oxidants are Lipochroman-6, sodium ascorbylphosphate, or combinations thereof. Preferably the compositions contain an amount of anti-oxidant of about 0.10% to about 4.0% by weight of the total composition. In a preferred embodiment Lipochroman-6 and sodium ascorbylphosphate are used. Preferably the compositions contain from about 0.01% to about 1.0% by weight of Lipochroman-6, and from about 0.10% to about 3.00% by weight of sodium ascorbylphosphate.

The compositions and medicaments according to the present invention preferably comprise a components selected from the group of sulfa drugs that are used to treat bacterial and some fungal infections. Suitable sulfa drugs comprise prontosil, sulfadiazine, sulfamethizole (Thiosulfil Forte®), sulfainethoxazole (Gantanol®), sulfasalazine (Azulfidine®), sulfisoxazole (Gantrisin®), and various high-strength combinations of three sulfonamides. Preferably, the sulfa drug is sulfadiazine.

The compositions and medicaments according to the present invention further preferably comprise a zinc component which are beneficial in wound healing. A suitable example is zinc gluconate.

The compositions and medicaments according to the present invention further preferably comprise an agent that promotes degradation of biofilms on open wounds. Suitable agents include peroxide forming enzymes such as lactoperoxidase as is disclosed in WO 88/02600 of Poulson, incorporated by reference, and glycoproteins such as lactoferrin as disclosed in EP A 1.545.587, incorporated by reference.

The compositions according to the present invention can optionally further comprise any pharmaceutically acceptable excipient, such as, for example, colorants, (de)odorants, preservatives and the like. The composition, according to the present invention, is intended for use in the treatment of open wounds and burns. The term “open wound”, as used herein, may refer to any type of tissue injury, but particularly to tissue injuries characterised by delay or complete failure of healing. Typical but non-limiting examples of such injuries are traumatic injury, including burns, injury resulting from surgery, diabetic wounds, pressure ulcers, arterial ulcers, decubitus ulcers, and venous stasis ulcers. The greatest benefits are achieved in injured tissues with compromised blood flow and oxygen supply.

The treatment of open wounds and burns according to the present invention typically comprises topical administration of the medicament or of a combination of the medicaments, containing the composition, to the open wound or burn. The medicament is preferably applied to the wounds or burns in amounts sufficient to completely cover the entire surface of the wound. In a preferred embodiment, the composition is applied to the open wound or burn, 1 to 8 times daily, more preferably 2 to 4 times daily. The treatment is continued as long as necessary to completely heal the wounds, it is applied to, or as long as beneficial effects are observed.

Although the aforementioned method of treatment generally applies, it is within the skill and within the objective of any professional, trained in the art of wound healing, to adjust the preferred amounts of the medicament and/or the frequency it is applied with, as well as the duration of the treatment, in order to optimise the efficacy for each individual patient.

In another embodiment of the present invention the composition further comprises an oxygen donor stabilising agent (component (c)). Addition of an oxygen donor stabilising agent will result in a composition, which releases the active component in a more controlled manner and which shows a longer lasting effect. Compositions containing the oxygen stabilising agent will further have improved stability during storage and transport under normal conditions. Preferably the oxygen donor stabilising agent is selected from the group consisting of organic acids or their (monovalent or polyvalent) pharmaceutically acceptable salts, preferably inorganic salts wherein the cations of the salts are preferably metals selected from groups 1 or 2 of the Periodic System of the Elements, or from the group consisting of saccharides.

The organic acids are preferably selected from the group consisting of chelating organic acids. The chelating organic acids are preferably selected from carboxylic acids containing one or more hydroxy and/or amino groups or from polycarboxylic acids optionally containing one or more hydroxy and/or amino groups.

Alternatively but depending on the intended use of the composition according to the invention, the chelating organic acids may be selected from polyphosphonic acids or their pharmaceutically acceptable salts as disclosed in U.S. Pat. No. 6,265,444 which is herein incorporated by reference, although then it is preferred that the polyphosphonic acid is used in combination with a carboxylic acid containing one or more hydroxy and/or amino groups or polycarboxylic acid optionally containing one or more hydroxy and/or amino groups such as EDTA. In this patent application a polyphosphonic acid is to be understood as a compound containing at least two —PO₃H₂ moieties or the pharmaceutical acceptable salt thereof. Preferably, the polyphosphonic acid has either formula I or formula II,

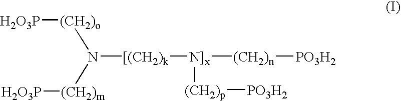

wherein x is an integer of 0 to 3 and k, m, n, o and p are each independently an integer of 1 to 4. Preferably, x is 2 and k, m, n, o and p are each 1 or 2;

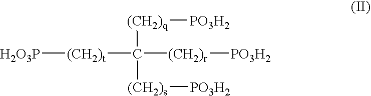

wherein q, r, s and t are each independently an integer of 0 to 4. Preferably q, r, s and t are 0 or 1, and most preferably all are zero.

According to the invention, it is most preferred that the polycarboxylic acids contain one or more hydroxy and/or amino groups and such acids are generally known as hydroxypolycarboxylic acids and aminopolycarboxylic acids. Very suitable examples of the oxygen donor stabilising agents according to the invention are gluconic acid, ethylenediaminotetraacetic acid (EDTA), nitrilotriacetic acid (or 1,1′,1″tricarboxytrimethylamine) and methylglycinediacetic acid or their salts. Most particular the oxygen donor stabilising agent is sodium gluconate.

The saccharides are preferably monosaccharides or disaccharides. Preferred examples are glucose, fructose, sucrose, maltitol and lactitol.

According to the invention, the molar ratio of component (b) to the oxygen donor stabilising agent is preferably 0.1-5.0 (b) 1.0 oxygen donor stabilising agent. More preferably, this molar ratio is 0.1-5.0 (b) 1.0 oxygen donor stabilising agent and in particular 0.1-5.0 (b):1.0 oxygen donor stabilising agent.

Accordingly the molar ratio of component (a) to the oxygen donor stabilising agent is preferably 1.0-30.0 (a):1.0 oxygen donor stabilising agent. More preferably, this molar ratio is 5.0-25.0 (a):1.0 oxygen donor stabilising agent and in particular 10.0-20.0 (a):1.0 oxygen donor stabilising agent.

In the preferred embodiment of the present invention where the composition comprises the oxygen donor stabilising agent, the composition is used for the treatment of open wounds and burns, wherein the method comprises topically administering an amount of the medicament or of a combination of the medicaments, containing the composition, to the open wound or burn. The medicament is applied to the wounds or burns in amounts that are sufficient to completely cover the entire surface of the wound. In a preferred embodiment, the composition is applied to the open wound or burn, 1 to 8 times daily, more preferably 2 to 4 times daily. The treatment is continued as long as necessary to completely heal the wounds, it is applied to, or as long as beneficial effects are observed.

The medicament according to the present invention may be used in combination with any other known method or means for treating wounds and burns, as can be determined by any skilled professional in the art.

The invention is further illustrated by the following examples which are not intended to limit the scope of protection conferred by the claims.

### EXAMPLE 1

A wound gel and a wound spray were prepared according to the present invention, which can be used for the treatment of open wounds and burns by topical application. The compositions of the wound gel and wound spray are shown in table 1.

TABLE 1

| | wound gel | wound spray |
|---|---|---|
| water | 51.5% | 56.5% |
| Comp. (a) | 2.5% | 2.5% |
| Comp (b) | 1.72% | 1.72% |
| Comp. (c) | 1.0% | 1.0% |
| Wacker HDK20 | 5.00% | 0% |
| (liquid binder) | | |
| PEG 1500 (liquid | 0.1% | 0% |
| binder) | | |
| sucrose | 2.0% | 2.0% |
| ethanol (99.9%) | 20.0% | 20.0% |
| glycerine | 15.000% | 15.000% |
| cellulose gum | 0.6% | 0.600% |
| citric acid•H₂O | 0.6% | 0.63% |

### EXAMPLE 2

In this experiment, an open wound on the lateral side of the left lower leg, which had arisen spontaneously in a diabetic patient, was treated by chemical sympathectomy and by topical application of wound gel from Example 1, following a dosing scheme that was assessed, and adjusted when deemed necessary by a skilled healthcare professional. It is generally known that the healing process of diabetic ulcers can last up to several months with the currently known treatments, and even up to longer periods without treatment. After two weeks of treatment the wound was re-examined and an apparent improvement was seen; the wound surface had become cleaner. The treatment with the wound gel of the present invention was continued. In the following weeks the appearance of the wound became cleaner and after 14 weeks of treatment with the wound gel an apparent decrease in wound diameter became observable. The wound diameter had decreased substantially when re-examined after 6 months of treatment. The treatment with the wound gel was continued. During treatment the patient was re-examined several times and during each examination the wound was photographed in the exact same position each time, so as to obtain a series of photographs of the wound during the whole period of treatment that could be used to calculate surface of the wound (expressed in the amount of pixels that were comprised by the wound on each photograph) and wound size reduction. In table 2 the healing process is represented as the amount of pixels comprised by the wound on each photograph, the reduction of the size of the wound surface (amount of pixels), and the relative size of the wound on each moment, as a percentage of the initial size of the wound surface.

TABLE 2

| | | decrease of | |
|---|---|---|---|
| | | wound surface | percentage of |
| | wound surface | compared to | the wound surface |
| examination | (pixels) | day 0 (pixels) | on day 0 |
| day 0 | 85,608 | 0 | 100 |
| day 14 | 82,720 | 2,888 | 96.62648 |
| day 70 | 93,184 | −7,576 | 108.8496 |
| day 97 | 81,295 | 4,313 | 94.96192 |
| day 136 | 81,184 | 4,424 | 94.83226 |
| day 188 | 66,792 | 18,816 | 78.02075 |
| day 259 | 28,314 | 57,294 | 33.07401 |

### EXAMPLE 3

An injury in the lower leg of a patient, which had become necrotic and contained haematoma, could not be treated with flammazine (Merck Index 13^{th} Ed., page 1586, nr. 8988 (2001)), which is at present one of the common medicaments for treating wounds such as ulcers and burns, because of an allergy. Therefore it was treated with the wound gel, according to Example 1, by filling the wound with the gel twice daily. After two weeks of treatment according to this regimen, re-examination revealed that the wound surface had decreased significantly and had a clean, normal appearance. Quantitative evaluation of photographs of the wound at day 0 and day 14, revealed that the wound surface had decreased with 76%, within the two weeks of treatment. It is generally known that the healing of necrotic injuries, with haematoma, can last up to several months when treated with the presently common methods.

### EXAMPLE 4

A group of seven patients suffering from chronic and/or slow-healing wounds from various causes were treated with a composition according to the present invention. The composition was applied as a gel or a spray, according to example 1, following a regimen as encompassed by the present invention, tailor made to meet the specific needs and requirements of each individual case, as assessed by professional healthcare personnel. During the period of treatment the wounds were reexamined periodically. During each examination the wounds were photographed in the exact same position each time, so as to obtain a series of photographs of each wound that could be used to calculate surface of the wound (expressed in the amount of pixels that were comprised by the wound on each photograph) and wound size reduction. The results of these experiments are summarized in Table 3.

### EXAMPLE 5

A spina bifida patient that had been suffering from an open wound on the heel of the right foot for more than six months, entered the present study. The subject had been treated with flammazine, which is presently one of the common methods of treating such wounds. Initially this had resulted in a noticeable decrease of the surface of the wound and an improving appearance. However, after several weeks the healing process of the wound ceased with this treatment. On entering the study, the patient was switched to a combined regimen of a wound gel according to the present invention and a flammazine ointment, applied topically to the ulcer on the heel of the right foot twice daily in such amounts that it completely covered the entire surface of the wound. After 114 days of treatment the wound had completely closed.

TABLE 3

| | | | initial wound | wound surface | | |
|---|---|---|---|---|---|---|
| | | | surface | (pixels) after | difference | % |
| patient | description of the wound | treatment | (pixels) | treatment | (pixels) | reduction |
| 1 | chronic venous insufficiency | gel, | 9,702 | closed | 9,702 | 100% |
| | right foot/ankle, lateral side, | 196 days | | | | |
| | diabetic patient | | | | | |
| 2 | ulcus with necrosis, left leg, | gel, | 19,300 | closed | 19,300 | 100% |
| | lateral side | 260 days | | | | |
| 3 | decubitus on the left heel | spray, | 31,390 | 22,477 | 21,562 | 68.7% |
| | | 182 days | | | | |
| 4 | chronic ulcer, on the right | gel, | 28,728 | 22,477 | 6,251 | 21.8% |
| | lower leg, resulting from | 126 days | | | | |
| | trauma | | | | | |
| 5 | chronic wound with necrosis, | gel, | 42,746 | 35,179 | 28,326 | 66.3% |
| | on the left leg, resulting from | 133 days | | | | |
| | surgery | | | | | |
| 6 | chronic venous ulcer on the left | gel, | 50,702 | closed | 50,702 | 100% |
| | lower leg with sclerosis | 126 days | | | | |
| 7 | defect of the forefoot after | gel, | 37,742 | 15,510 | 22,232 | 58.9% |
| | amputation, diabetic patient | 331 days | | | | |

1-29. (canceled)

30. A method of treating a patient having an open wound and/or burn, comprising administering to the patient a composition comprising the following components:
(a) a salt selected from the group consisting of cations Aⁿ⁺ and anions derived from halogen oxides according to the general formula [OₘX]⁻, wherein A is a metal selected from Groups 1 or 2 of the Periodic System of the Elements, X is a halogen atom, m=1-4, n=1 or 2;
(b) an oxygen donor; and
(c) a liquid binder.

31. The method according to claim 30, wherein the oxygen donor is selected from the group consisting of metal perborate compounds, metal peroxide compounds, metal percarbonate compounds, metal persulfate compounds, metal perphosphate compounds, wherein the metal is an alkaline or alkaline-earth metal, halogen oxide compounds, hydrogen peroxide, and organic peroxides.

32. The method according to claim 31, wherein the oxygen donor is a metal perborate compound, a metal percarbonate compound, a metal peroxide compound, a hydrogen peroxide, a halogen oxide compound, an organic peroxide, or a combination thereof.

33. The method according to claim 32, wherein the metal perborate compound is a salt selected from the group consisting of cations Aⁿ⁺ and anions derived from borates according to the general formula [BₚO_{q}]^{r-}, wherein A is a metal selected from Groups 1 or 2 of the Periodic System of the Elements; p=1-4; q=1-8; and r=1-3.

34. The method according to claim 30, wherein A is lithium, sodium or potassium; n=m=p=1; q=2 or 3; and r=1-3.

35. The method according to claim 30, wherein A is sodium, X is chlorine, p=1, q=3 and r=1.

36. The method according to claim 32, wherein the metal peroxide compound is calcium peroxide or magnesium peroxide.

37. The method according to claim 32, wherein the halogen oxide compound is chlorine oxide.

38. The method according to claim 30, wherein component (a) comprises sodium hypochlorite.

39. The method according to claim 32, wherein the metal perborate compound is sodium perborate.

40. The method according to claim 30, wherein the liquid binder is a liquid polyol, a polymeric binder, fumed silica, a gum, or a combination thereof.

41. The method according to claim 30, wherein the composition further comprises a gelatinous thickener.

42. The method according to claim 41, wherein the gelatinous thickener is a cellulose material.

43. The method according to claim 30, wherein the composition further comprises an agent that counteracts loss of moisture.

44. The method according to claim 43, wherein the agent that counteracts loss of moisture is a carbohydrate.

45. The method according to claim 30, wherein the composition further comprises an anti-oxidant.

46. The method according to claim 30, wherein the administering comprises topical application of the composition 2-4 times a day.

47. The method according to claim 30, wherein the composition further comprises an oxygen donor stabilizing agent.

48. The method according to claim 47, wherein the oxygen donor stabilising agent is an organic acid, a salt thereof, a saccharide, or a combination thereof.

49. The method according to claim 48, wherein the salt of an organic acid is an inorganic salt.

50. The method according to claim 49, wherein cations of the salt are metals selected from Groups 1 or 2 of the Periodic System of Elements.

51. The method according to claim 48, wherein the organic acid is a chelating organic acid.

52. The method according to claim 48, wherein the organic acid is a carboxylic acid having one or more hydroxy and/or amino groups; a polycarboxylic acid, optionally having one or more hydroxy and/or amino groups; or both.

53. The method according to claim 52, wherein the polycarboxylic acid is a hydroxypolycarboxylic acid or a aminopolycarboxylic acid.

54. The method according to claim 47, wherein the molar ratio of component (b) to the oxygen donor stabilizing agent is 0.1-5.0 (b):1.0 oxygen donor stabilizing agent.

55. The method according to claim 47, wherein the molar ratio of component (a) to the oxygen donor stabilizing agent is 10.0-20.0 (a):1.0 oxygen donor stabilizing agent.

56. The method according to claim 30, wherein the composition is a gel, a spray, or dressing.

57. A wound dressing comprising the liquid composition comprising the following components:
(a) a salt selected from the group consisting of cations Aⁿ⁺ and anions derived from halogen oxides according to the general formula [OₘX]⁻, wherein A is a metal selected from Groups 1 or 2 of the Periodic System of the Elements, X is a halogen atom, m=1-4, n=1 or 2;
(b) an oxygen donor; and
(c) a liquid binder.

58. A method of treating a patient having an open wound and/or burn, comprising administering to the patient a composition being obtainable by combining the following components:
(a) a salt selected from the group consisting of cations Aⁿ⁺ and anions derived from halogen oxides according to the general formula [OₘX]⁻, wherein A is a metal selected from Groups 1 or 2 of the Periodic System of the Elements, X is a halogen atom, m=1-4, n=1 or 2;
(b) an oxygen donor; and
(c) a liquid binder.
